(19) United States
(12) Patent Application Publication
(10) Pub. No.: US 2011/0274656 A1
(43) Pub. Date: Nov. 10, 2011

(54) PHARMACEUTICAL COMPOSITION FOR PREVENTING OR TREATING HEPATITIS C, COMPRISING THE ROOTS EXTRACT OF PLATYCODON GRANDIFLORUM OR PLATYCODON GRANDIFLORUM SAPONIN COMPONENTS

(75) Inventors: Jong Woo Kim, Gyeonggi-do (KR); Sang Wook Lee, Gyeonggi-do (KR); Sang Jin Park, Gyeonggi-do (KR); Jung Cheul Shin, Gyeonggi-do (KR); Jae Won Yang, Gyeonggi-do (KR); Jong Hwan Lim, Daejeon (KR)

(73) Assignee: B&C Biopharm Co., Ltd., Suwon-si (KR)

(21) Appl. No.: 13/143,895

(22) PCT Filed: Jan. 21, 2010
(86) PCT No.: PCT/KR10/00376

§ 371 Date: Jul. 8, 2011

(30) Foreign Application Priority Data
Jan. 23, 2009 (KR) 10-2009-0006078
Oct. 21, 2009 (KR) 10-2009-0100092

(51) Int. Cl. A61K 38/21; C07H 15/24; C07C 69/757; C07C 62/34; A61P 31/14; A61K 31/704; A61K 31/215; A61K 31/19; A61K 31/366; A61K 36/346; C07D 311/00

(52) U.S. Cl. 424/85.5; 424/773; 424/85.4; 424/85.6; 424/85.7; 536/18.1; 560/116; 562/498; 549/275; 514/33; 514/510; 514/557; 514/453

(57) ABSTRACT

The present invention relates to a pharmaceutical composition for preventing or treating Hepatitis C, comprising the roots extract of Platycodon grandiflorum and/or saponin components in Platycodon grandiflorum useful as an antiviral agent. The composition of the present invention has no harm to human and inhibits the proliferation of Hepatitis C virus, so that it can be effectively used as a preventive or therapeutic agent for Hepatitis C.

### TECHNICAL FIELD

The present invention relates to saponin components of Platycodon grandiflorum useful as antiviral agents or the roots extract of Platycodon grandiflorum containing the same and a pharmaceutical composition comprising thereof for preventing or treating Hepatitis C.

### BACKGROUND ART

Hepatitis C virus (referred as “HCV” hereinafter) is transferred via transfusion and community-acquired infection. Once infected with HCV, 20% of the infected patients are developed into acute hepatitis and about 80% are suffered by chronic hepatitis which will be possibly developed into liver cirrhosis or liver cancer. According to recent reports, approximately 200 million people are infected with HCV world-widely and 4.5 million people are presumed to be infected with HCV in USA (it is assumed that the number could be grown to 15 million). In Europe, at least 5 million people are presumed to be Hepatitis C patients.

Satisfactory vaccine against Hepatitis C or an effective therapeutic agent treating Hepatitis C has not been developed, yet. Therefore, numbers of pharmaceutical companies and research institutes all over the world have been trying to develop an effective Hepatitis C treating agent. Compared with Hepatitis B, HCV patients are prevalent across the world and demonstrate much higher potential for liver cirrhosis and liver cancer. In addition, Hepatitis C has higher potential for chronic hepatitis, and the mechanism for such progress has been still studied. Hepatitis C virus is transferred not only by transfusion but also by intravenous drug injection or printing tattoo, but mostly by direct blood contact. Once infected with HCV, most of the infected patients progress to chronic hepatitis and then further to liver cirrhosis and liver cancer. Therefore, it is urgently requested to develop an effective vaccine and a therapeutic agent to treat Hepatitis C. There are many different genotypes and mutations among HCV strains. So, when chronic hepatitis is developed by HCV infection, there is a high chance of re-infection or co-infection because of genetic variants. That makes HCV vaccine development difficult.

The current treatment for hepatitis C is combination therapy of Interferon-α with Ribavirin. But, this treatment demonstrates very low rate of cure and brings severe side effects. About 25% of Hepatitis C patients do not respond to Interferon-α and another 25% of patients are apt to have relapsed into the disease after temporary response. The rest 50% of patients maintain normal ALT level and remain HCV RNA negative even after treatment has been finished. But, 50% of those treated patients have relapsed into it in 3-6 months from the first treatment. Thus, only 25% of Hepatitis C patients show sustained viral response (SVR), which means treatment effect is retained at least 6 months in those patients. Meanwhile, Hepatitis C virus has 6 genotypes. Among them, genotype 1b is most common but does not respond to Interferon-α so well, compared with genotype 2 or genotype 3. In case of combination therapy with Interferon-α and Ribavirin, the treatment effect is double. When Ribavirin alone is treated, the treatment effect is not so good and rather brings side effects such as anemia resulted from erythroclasis. So, Ribavirin is prescribed only when a patient does not respond to Interferon-α or Hepatitis C is relapsed. So far, an effective antiviral agent that is specially targeted to hepatitis C virus by inhibiting the replication directly has not been developed yet.

RNA genome was first isolated from HCV by molecular cloning in 1989 (Choo, Q-L, et al., 1989, Isolation of a cDNA clone derived from a blood-borne non-A, non-B viral hepatitis genome. Science 244:359-362). Since then, molecular biological approaches to HCV have been made, which have been limited though because of lack of efficient cell culture system and animal model. But recently, a hepatoma cell line replicating HCV RNA replicon stably has been established to overcome the limitation (Lohmann, V., F. Korner, J-O Koch, U. Herian, L. Theilmann, R. Bartenschlager, 1999, Replication of subgenomic hepatitis c virus RNAs in a hepatoma cell line. Science 285:110-113). HCV RNA replicon is divided into two categories; full length replicon containing whole HCV gene and subgenomic replicon in which structural proteins are excluded. HCV RNA replicon is bicistronic replicon containing HCV 5 end, HCV IRES, neomycin resistant gene (neomycin transferase gene), and EMCV (encephalomyocarditis virus) IRES. HCV nonstructural proteins are composed of the sequences comprising NS3-NS5B and HCV 3′ end (untranslational region). HCV replicons against each genotype of HCV are developed, which help different case studies.

The present inventors completed this invention by suggesting that saponin components of Platycodon grandiflorum isolated from the roots extract of Platycodon grandiflorum and the roots extract of Platycodon grandiflorum containing the same can be effectively used for the prevention and/or treatment of Hepatitis C based on the confirmation by the inventors that saponin components of Platycodon grandiflorum isolated from the roots extract of Platycodon grandiflorum and the roots extract of Platycodon grandiflorum comprising the same has excellent HCV replicon inhibitory effect.

Platycodi Radix is the root of Platycodon grandiflorum A.DC., a perennial plant belonging to Campanulaceae, that was widely distributed or cultivated in East Asia, whose galenical name is Gilgyeong.

The roots of Platycodon grandiflorum are reported to contain lots of carbohydrate (sugar, at least 90%), protein (2.4%), lipid (0.1%) and ash (1.5%). Additionally, it contains various kinds of triterpenoid saponins (24 kinds including platycodin A, C, D, D₂; polygalacin D, D₂, etc) (about 2%). Those saponins have been paid much attentions because of their various pharmacological effects, making them active ingredients of Platycodon grandiflorum. Other minor ingredients of Platycodon grandiflorum are also reported such as steroid compounds such as α-spinasterol, Δ7-stigmasterol and α-spinasteryl-β-D-glucoside, which take 0.03%. Carbohydrate components in Platycodon grandiflorum are mainly consist of monosaccharides, disaccharides or trisaccharides such as glucose, fructose, saccharose, kestose, etc and some polysaccharides such as inulin and platycodinin are included as well.

Lots of pharmacological effects of Platycodongrandiflorum that have been proved by modern medicinal studies are as follows, protecting brain cells [Yoo Ki-Yeon et al., Neurosci. Lett. 444(1), 97-101 (2008)], anti-obesity [Zhao, H. L. et al., J. Food. Sci. 73(8), H195-H200, (2008)], protecting liver functions [Lee, K. J. et al., Toxicol. Lett. 147, 271-282, (2004)], regulating immune functions [Ahn, K. S. et al., Life Sci. 76, 2315-2328, (2005)] and causing cytotoxicity [Lee, Kyung Jin et al., Food Chem. Toxicol. 46(5), 1778-1785 (2008); Zhang, Lin et al., Molecules 12(4), 832-841, (2007)].
[Reference 1] Yoo Ki-Yeon. et al., Neurosci. Lett. 444(1), 97-101, (2008)
[Reference 2] Zhao, H. L. et al., J. Food Sci. 73(8), H195-H200, (2008)
[Reference 3] Lee, K. J. et al., Toxicol. Lett. 147, 271-282, (2004)
[Reference 4] Ahn, K. S. et al., Life Sci. 76, 2315-2328, (2005)
[Reference 5] Lee, K. J. et al., Food Chem. Toxicol. 46(5), 1778-1785, (2008)
[Reference 6] Zhang, L. et al., Molecules 12(4), 832-841, (2007)

### DISCLOSURE OF INVENTION

### Technical Problem

It is an object of the present invention to provide a pharmaceutical composition effective for preventing and treating Hepatitis C.

### Solution to Problem

To achieve the above object, the present invention provides a pharmaceutical composition comprising some saponin components of Platycodon grandiflorum isolated from the roots extract of Platycodon grandiflorum and the roots extract of Platycodon grandiflorum containing the same as an active ingredient and a pharmaceutically acceptable carrier for preventing and treating Hepatitis C.

The pharmaceutical composition of the present invention comprising saponin components of Platycodon grandiflorum isolated from the roots extract of Platycodon grandiflorum and the roots extract of Platycodon grandiflorum containing the same as an active ingredient and a pharmaceutically acceptable carrier for preventing and treating Hepatitis C can be treated alone as a preventive or a therapeutic agent for Hepatitis C or treated with interferon and/or Ribavirin, or treated in combination with a mixed composition of one or at least two kinds of compounds selected from all kinds of Hepatitis C virus proliferation inhibitors including immune modulators, cell signaling regulators, antiviral agents, HCV polymerase (NS5B) inhibitors, HCV protease (NS3/4A) inhibitors, HCV helicase (NS3 helicase) inhibitors, HCV NS4B inhibitors, HCV NS5A inhibitors, HCV cell entry inhibitors, and HCV assembly inhibitors.

Interferon herein includes every kind of interferon, which is exemplified by natural interferon, Interferon-α, Interferon-β, Interferon-γ, pegylated-Interferon, albumin-linked Interferon, etc, and is preferably one or more interferons selected from the above, but not always limited thereto.

Antiviral agent herein is one or more drugs selected from the group consisting of Ribavirin, Lamivudine, Amantadine, etc, but not always limited thereto.

In a preferred embodiment of the present invention, the invention provides a pharmaceutical composition comprising one or more substances selected from the group consisting of Platycodon grandiflorum extract extracted by using water, organic solvent or a mixture thereof; and saponin components of Platycodon grandiflorum isolated from the roots extract of Platycodon grandiflorum and the roots extract of Platycodon grandiflorum containing the same as an active ingredient for preventing or treating Hepatitis C.

The organic solvent herein is preferably 10-100% concentration of C₁-C₄ lower alcohol.

The said Platycodon grandiflorum extract includes such Platycodon grandiflorum extract purified by ultra-filtration membrane from the Platycodon grandiflorum extract prepared by solvent extraction.

At this time, ultra-filtration membrane with the molecular weight cut-off of 100,000, 5,000 or 1,000 is used. So, the Platycodon grandiflorum extract purified by the ultra-filtration membrane is the extract having the molecular weight of 1,000-100,000 or the extract having the molecular weight of 1,000-5,000.

The said Platycodon grandiflorum extract includes the extract having the molecular weight of 500-5,000 isolated from the Platycodon grandiflorum extract purified by ultra-filtration membrane by using nano-filtration membrane with the molecular weight cut-off of up to 500.

The saponin compound isolated from Platycodon grandiflorum extract includes the saponin compound represented by formula 1 and the (pro)sapogenin compound represented by formula 2:

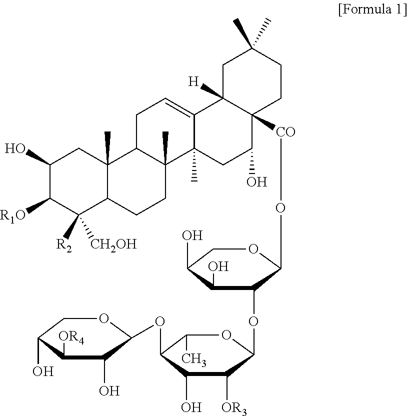

In formula 1,

R₁ is glucopyranosyl-(1→6)-glucopyranosyl-(1→6)-glucopyranosyl

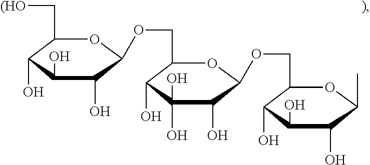

gentiobiosyl

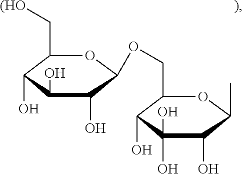

glucosyl

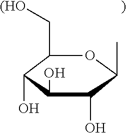

or laminaribiosyl

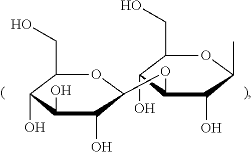

R₂ is CH₂OH, CH₃, COOH or COOCH₃,

R₃ is H or acetyl,

R₄ is H or apiosyl

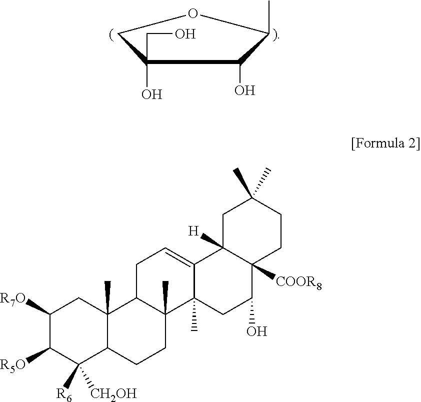

In formula 2,

R₅ is H, glucosyl or laminaribiosyl,

R₆ is CH₂OH or CH₃,

R₇ is H,

R₆ and R₇ can be —CO— linked each other,

R₈ is H or CH₃.

The saponin compound represented by formula 1 is exemplified by deapioplatycoside E, platycoside E, platycodin D₃, polygalacin D₂, polygalacin D, platyconic acid A, deapioplatycodin D₂, platycodin D₂, deapioplatycodin D, platycodin D, 2″-O-acetyl-deapiopolygalacin D₂, 2″-O-acetyl-polygalacin D₂ or platyconic acid A methyl ester.

The (pro)sapogenin compound represented by formula 2 is exemplified by the group consisting of platycodigenin, polygalacic acid, platycogenic acid A lactone, platycogenic acid A lactone 3-O-glucopyranoside, platycodigenin 3-O-glucopyranoside 28-methyl ester or platycodigenin 3-O-laminaribioside 28-methyl ester.

The saponin components include the Platycodon grandiflorum crude saponin which was prepared as follows: first, the root extract of Platycodon grandiflorum was purified by using reverse phase column chromatography, which was dissolved in ethanol or methanol, followed by precipitation with ethyl acetate, resulting in Platycodon grandiflorum crude saponin. The (pro)sapogenin compound represented by formula 2 is prepared by hydrolysis of the Platycodon grandiflorum crude saponin.

As explained hereinbefore, the pharmaceutical composition for preventing or treating Hepatitis C of the present invention can additionally include one or more HCV proliferation inhibitors.

The HCV proliferation inhibitor is selected from the group consisting of immune modulator, cell signalling regulator, antiviral agent, HCV polymerase (NS5B) inhibitor, HCV protease (NS3/4A) inhibitor, HCV helicase (NS3 helicase) inhibitor, HCV NS4B inhibitor, HCV NS5A inhibitor, HCV cell entry inhibitor and HCV assembly inhibitor.

The pharmaceutical composition for preventing or treating Hepatitis C of the present invention can also include interferon which is the immune modulator; and Ribavirin.

The immune modulator herein is selected from the group consisting of natural interferon, Interferon-α, Interferon-β, Interferon-γ, pegylated-Interferon, albumin-linked Interferon and cytokine.

The present invention also provides a method of combination therapy of the pharmaceutical composition comprising the roots extract of Platycodon grandiflorum for preventing or treating Hepatitis C and the said HCV proliferation inhibitor.

It is more preferred to co-administrate the pharmaceutical composition the roots extract of Platycodon grandiflorum for preventing or treating Hepatitis C with an immune modulator and Ribavirin altogether.

The present invention further provides a health functional food for the prevention of Hepatitis C or improvement of Hepatitis C treatment, which comprising one or more substances selected from the group consisting of the roots extract of Platycodon grandiflorum extracted by using water, organic solvent or a mixture thereof; and saponin components isolated from the Platycodon grandiflorum.

### Advantageous Effects of Invention

Saponin components of Platycodon grandiflorum isolated from the roots extract of Platycodon grandiflorum and the roots extract of Platycodon grandiflorum containing the same and a composition comprising thereof as an active ingredient have no harm to human and inhibit HCV proliferation effectively, so that they can be effectively used as preventive or therapeutic agents for Hepatitis C.

### BEST MODE FOR CARRYING OUT THE INVENTION

Hereinafter, the present invention is described in detail.

The roots extract of Platycodon grandiflorum of the present invention can be prepared by extracting Platycodon grandiflorum with water, organic solvent or a mixture thereof. At this time, the organic solvent is C₁-C₄ alcohol such as methanol or ethanol, ethyl acetate, hexane and dichloromethane. Particularly, 0-100% concentration of C₁-C₄ alcohol aqueous solution is preferred and 0-100% concentration of ethyl alcohol (alcohol spirit) aqueous solution is more preferred.

The roots extract of Platycodon grandiflorum of the present invention can be prepared by extracting the raw roots of Platycodon grandiflorum, dried one or pulverized one with a solvent.

Particularly, the dried roots of Platycodon grandiflorum was pulverized with blender and soaked in 2-200 times volume of water or an organic solvent, more preferably in 10-30 times volume, followed by extraction at 10-100° C. The extraction can be performed by immersion extraction, ultrasonic extraction, or reflux extraction. If necessary, extraction is repeated more than two times. The obtained extract is filtered or centrifuged to eliminate solid contents, followed by concentration and freeze-drying. As a result, completely dried Platycodon grandiflorum solvent extract is prepared.

Isolation and purification of Platycodon grandiflorum crude saponin from the roots extract of Platycodon grandiflorum was performed as follows: the roots extract of Platycodon grandiflorum was suspended in distilled water of 5-50 times weight of the extract, and poured into a column packed with reverse phase gel (RP-18, Diaion HP-20, MCI-gel, etc) or ion exchange gel of 5-100 times weight of the extract. The column was further washed with additional distilled water of 50-1000 times weight of the extract to eliminate non-absorbed sugar and amino acid, etc. After washing with water, the column was eluted with 10-100 times weight of aqueous alcohol, and the eluates were pooled up and concentrated to dryness, which was dissolved in 10-50 times weight of alcohol and filtered. The filtrate was concentrated to dryness to give the crude saponin of Platycodon grandiflorum.

Isolation and purification of saponin components of Platycodon grandiflorum from the roots extract of Platycodon grandiflorum or the Platycodon grandiflorum crude saponin is as follows: the crude saponin is dissolved in water of 5-20 times weight, followed by isolation and purification using MPLC or HPLC equipped with column filled with reverse phase gel (RP-18, MCI-gel, etc).

The pharmaceutical composition of the present invention can include saponin components of Platycodon grandiflorum or the roots extract of Platycodon grandiflorum at the concentration of 0.1-90 weight % and more preferably 10-70 weight %.

The pharmaceutical composition comprising saponin components of Platycodon grandiflorum or the roots extract of Platycodon grandiflorum containing the same inhibit HCV proliferation significantly, suggesting excellent preventive or treatment effect on Hepatitis C.

The pharmaceutical composition comprising saponin components of Platycodon grandiflorum or the roots extract of Platycodon grandiflorum containing the same of the present invention can additionally include a pharmaceutically acceptable carrier, an excipient and a diluent. The pharmaceutical composition comprising saponin components of Platycodon grandiflorum or the roots extract of Platycodon grandiflorum containing the same of the present invention can be formulated for oral administration, for example powders, granules, tablets, pills, capsules, solutions, suspensions, emulsions, and syrups. The carriers, expients and diluents are exemplified by lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate and mineral oil. Solid formulations for oral administration are tablets, pills, powders, granules and capsules. These solid formulations are prepared by mixing with one or more suitable excipients such as starch, calcium carbonate, sucrose or lactose, gelatin, etc. Except for the simple excipients, lubricants, for example magnesium stearate, talc, etc, can be used. Liquid formulations for oral administrations are suspensions, solutions, emulsions and syrups, and the above-mentioned formulations can contain various excipients such as wetting agents, sweeteners, aromatics and preservatives in addition to generally used simple diluents such as water and liquid paraffin.

The composition of the present invention contains not only saponin components of Platycodon grandiflorum or the roots extract of Platycodon grandiflorum containing the same but also any kind of HCV proliferation inhibitors or a mixture thereof exemplified by immune modulators such as interferon used to be administered for preventing or treating Hepatitis C, cell signaling regulators, antiviral agents such as Ribavirin, HCV polymerase (NS5B) inhibitors, HCV protease (NS3/4A) inhibitors, HCV helicase (NS3 helicase) inhibitors, HCV NS4B inhibitors, HCV NS5A inhibitors, HCV cell entry inhibitors, and HCV assembly inhibitors.

The pharmaceutical composition of the present invention can be administered by various pathways including oral, transdermal, hypodermic, intramuscular or intravenous administration. The effective dosage of the pharmaceutical composition of the present invention can be determined according to age, gender, weight and health condition of a patient and severity of a disease by those in the art. In the case of human, the pharmaceutical composition can be administered by 0.02-1000 mg/kg per day, and more preferably by 1-200 mg/kg per day. The administration frequency is once a day or a few times a day. The dosage cannot limit the scope of the present invention by any means.

### MODE FOR THE INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Preparation of Platycodon grandiflorum Extract

5,000 ml of distilled water was added to 1,000 g of pulverized roots of Platycodon grandiflorum, followed by reflux-extraction for 6 hours at 90? twice. After cooling down, the extract proceeded to centrifugation (10,000×g) for 30 minutes at room temperature to eliminate solid substances. Upon completion of the centrifugation, the solution was freeze-dried to give 368 g of Platycodon grandiflorum extract powder (DrJ-1).

5,000 ml of ethanol (alcohol spirit) was added to 1,000 g of pulverized roots of Platycodon grandiflorum, followed by reflux-extraction in water bath for 6 hours twice. After cooling down, the extract proceeded to centrifugation (10,000×g) for 30 minutes at room temperature to eliminate solid substances. Upon completion of the centrifugation, the solution was dried under reduced pressure to give 136 g of Platycodon grandiflorum extract powder (DrJ-2).

5,000 ml of methanol was added to 1,000 g of pulverized roots of Platycodon grandiflorum, followed by reflux-extraction in water bath for 6 hours twice. After cooling down, the extract proceeded to centrifugation (10,000×g) for 30 minutes at room temperature to eliminate solid substances. Upon completion of the centrifugation, the solution was dried under reduced pressure to give 205 g of Platycodon grandiflorum extract powder (DrJ-3).

Mixture of ethanol (alcohol spirit) and water at the ratio as shown in Table 1 were added to 1,000 g of pulverized roots of Platycodon grandiflorum to make the volume of each solution to be 5,000 ml, followed by reflux-extraction in water bath for 6 hours twice. After cooling down, the extract proceeded to centrifugation (10,000×g) for 30 minutes at room temperature to eliminate solid substances. Upon completion of the centrifugation, the solution was dried under reduced pressure to give Platycodon grandiflorum extract powder as follows.

Table 1

TABLE 1

| | | | Amount of the roots extract of |
|---|---|---|---|
| Sample | Water | Alcohol Spirit | Platycodon grandiflorum |
| DrJ-4 | 10% | 90% | 156 g |
| DrJ-5 | 25% | 75% | 175 g |
| DrJ-6 | 50% | 50% | 230 g |
| DrJ-7 | 75% | 25% | 305 g |
| DrJ-8 | 90% | 10% | 356 g |

### Example 2

### Separation of Crude Saponin from the Roots Extract of Platycodon grandiflorum

### Preparation Example 1

### Separation of Crude Saponin from Platycodon grandiflorum Water Extract

Crude saponin was separated from the Platycodon grandiflorum water extract (DrJ-1) prepared in Example 1 by the method described below.

100 g of Platycodon grandiflorum water extract (DrJ-1) was dissolved in 1,000 ml of water, which was loaded in the column (50 250 mm) filled with 500 ml of reverse phase gel (HP-20, RP-18, or MCI gel) to let crude saponin absorbed. To eliminate sugars which were not absorbed (glucose, sorbitol, fructose, sucrose and inulin such as fructooligosaccharide), 1,000 ml of water and 500 ml of 3-5% acetonitrile aqueous solution were running and 500 ml of water was running again to eliminate acetonitrile. When sugars were completely eliminated, 30-90% ethanol aqueous solution (500 ml) was spilled enough to take off the absorbed components. The ethanol aqueous solution was distilled under reduced pressure. As a result, 7 g of solid content was obtained. 50 ml of ethanol was added to the obtained solid content and those not dissolved in ethanol were filtered out. 100 ml of ethyl acetate was added to the filtrate and then generated solid was filtered and dried to give 6.5 g of crude saponin (DrJ-9).

### Preparation Example 2

### Separation of Crude Saponin from Platycodon grandiflorum Ethanol Extract

Crude saponin was separated and purified from 100 g of the Platycodon grandiflorum ethanol (alcohol spirit) extract (DrJ-2) prepared in Example 1 by the same manner as described in Preparation Example 1 of Example 2. As a result, 8 g of crude saponin (DrJ-10) was obtained.

### Preparation Example 3

### Separation of Crude Saponin from Platycodon Grandiflorum Methanol Extract

Crude saponin was separated and purified from 100 g of the Platycodon grandiflorum methanol extract (DrJ-3) prepared in Example 1 by the same manner as described in Preparation Example 1 of Example 2. As a result, 9.8 g of crude saponin (DrJ-11) was obtained.

### Example 3

### Preparation of Platycodon grandiflorum Compositions by Ultra-Filtration

### Preparation Example 1

### Preparation of Composition Containing Platycodon grandiflorum Saponin from Water Extract by Filtration Using Ultra-Filtration Membrane

Pellicon 2 TFF system (Millipore USA, PART# xi42 pm001) was used as an ultrafiltration membrane. 100 g of the Platycodon grandiflorum water extract (DrJ-1) prepared in Example 1 was dissolved in 18,000 ml of distilled water, which was filtered through the ultra-filtration membrane (Pellicon 2, 100 KDa) until the residue was reached to the volume of 100 ml. Additional 1,000 ml of water was added to the residue, which was filtered through the ultra-filtration membrane (Pellicon 2, 100 KDa) again until the volume of the residue reached 100 ml or less. The filtrate was concentrated under reduced pressure to give 80 g of Platycodon grandiflorum composition (DrJ-12) having the molecular weight less than 100,000. 70 g of DrJ-12 (Platycodon grandiflorum composition having the molecular weight less than 100,000) was dissolved in 16,000 ml of water, which was filtered through the ultra-filtration membrane (Pellicon 2, 5 KDa) by the same manner as described above. As a result, 24 g of Platycodon grandiflorum composition having the molecular weight less than 5,000 (DrJ-13) was obtained. 14 g of DrJ-13 (Platycodon grandiflorum composition having the molecular weight less than 5,000) was dissolved in 6,000 ml of water, which was filtered through the ultra-filtration membrane (Pellicon 2, 1 KDa) until the volume of the residue reached 100 ml. Additional 1,000 ml of water was added to the remnant and filtered through the ultra-filtration membrane (Pellicon 2, 1 KDa) again until the volume of the residue reached 100 ml or less. The final residue which did not passed through the ultra-filtration membrane (Pellicon 2, 1 KDa) was concentrated under reduced pressure to give 10.4 g of Platycodon grandiflorum composition (DrJ-14) having the molecular weight of 1,000-5,000.

100 g of the Platycodon grandiflorum water extract (DrJ-1) prepared in Example 1 was filtered through the ultra-filtration membrane (Pellicon 2, 100 KDa) by the same manner as described above. The filtrate was filtered through the ultra-filtration membrane (Pellicon 2, 1 KDa) to give 15.6 g of Platycodon grandiflorum composition (DrJ-15) having the molecular weight of 1,000-100,000.

### Preparation Example 2

### Preparation of Composition Containing Platycodon grandiflorum Saponin from Ethanol Extract by Filtration Using Ultra-Filtration Membrane

The Platycodon grandiflorum ethanol (alcohol spirit) extract (DrJ-2) prepared in Example 1 was treated by the same manner as described in Preparation Example 1 of Example 3. As a result, 30 g of Platycodon grandiflorum composition (DrJ-16) having the molecular weight less than 100,000, 19.5 g of Platycodon grandiflorum composition (DrJ-17) having the molecular weight less than 5,000, 11.8 g of Platycodon grandiflorum composition (DrJ-18) having the molecular weight of 1,000-5,000, and 10.7 g of Platycodon grandiflorum composition (DrJ-19) having the molecular weight of 1,000-100,000 were obtained.

### Preparation Example 3

### Preparation of Platycodon grandiflorum Composition from Methanol Extract by Filtration Using Ultra-Filtration Membrane

The Platycodon grandiflorum methanol extract (DrJ-3) prepared in Example 1 was treated by the same manner as described in Preparation Example 1 of Example 3. As a result, 90 g of Platycodon grandiflorum composition (DrJ-20) having the molecular weight less than 100,000, 70 g of Platycodon grandiflorum composition (DrJ-21) having the molecular weight less than 5,000, 12.8 g of Platycodon grandiflorum composition (DrJ-22) having the molecular weight of 1,000-5,000 and 11.5 g of Platycodon grandiflorum composition (DrJ-23) having the molecular weight of 1,000-100,000 were obtained.

### Example 4

### Preparation of Composition Containing Platycodon grandiflorum Saponin from Water Extract by Filtration Using Nano-Filtration Membrane

8 g of DrJ-13 obtained from Platycodon grandiflorum water extract (DrJ-1) was dissolved in 10,000 ml of distilled water, which was passed through the nano-filtration membrane (molecular weight cut-off: 500, nano Filtration Process Scale, Low) until the volume of the residue reached 100 ml or less. Additional 1,000 ml of water was added to the residue, which was repeatedly passed through the nano-filtration membrane (molecular weight cut-off: 500, nano Filtration Process Scale, Low) until the volume of the residue reached 100 ml. The residue was concentrated under reduced pressure to give 5 g of Platycodon grandiflorum composition (DrJ-24) having the molecular weight of 500-5,000.

Nano-filtration was performed with DrJ-17 (ethanol extract) and DrJ-21 (methanol extract) having the molecular weight less than 5,000 by the same manner as described above and as a result Platycodon grandiflorum compositions DrJ-25 and DrJ-26 having the molecular weight of 500 5,000 were obtained.

### Example 5

### Isolation and Purification of Platycodon grandiflorum Saponin

According to the methods described in literatures published previously, Platycodon grandiflorum saponins such as deapioplatycoside E, platycoside E, platycodin D₃, polygalacin D₂, polygalacin D, platyconic acid A, deapioplatycodin D₂, platycodin D₂, deapioplatycodin D, platycodin D, 2″-O-acetyl-deapiopolygalacin D₂ and 2″-O-acetyl-polygalacin D₂, etc were isolated and purified [Kim, Y. S. et al., Planta Med. 71, 566-568, (2005); Choi, Y. H. et al., Molecules 13(11), 2871-2879, (2008)].

Particularly, 220 g of the methanol extract obtained in Example 1 was suspended in 2.2 L of distilled water, which was adsorbed on Diaion HP-20 column (Φ=5.0*100 cm), followed by washing with 10 L of distilled water. After wash, the column was eluted with equal amount of 20% methanol, 85% methanol, and methanol stepwise and as a result three fractions (fraction #1˜fraction #3) were obtained. Fraction #2 (eluted with 85% methanol) was loaded on Futecs NS-3000i system HPLC equipped with RP-18 column to separate 12 saponin compounds. At this time, 20 mM KH₂PO₄ and 26% acetonitrile were used as elution buffers. The saponin compounds were identified by spectroscopic data as follows: compound #1, deapioplatycoside E (Rₜ 25.18 min); compound #2, platycoside E (Rₜ 26.38 min); compound #3, platycodin D₃ (Rₜ 35.41 min); compound #4, polygalacin D₂ (Rₜ 41.28 min); compound #5, polygalacin D (Rₜ 44.06 min); compound #6, platyconic acid A (Rₜ 49.29 min); compound #7, deapioplatycodin D₂ (Rₜ 57.49 min); compound #8, platycodin D₂ (Rₜ 62.86 min); compound #9, deapioplatycodin D (Rₜ 62.08 min); compound #10, platycodin D (Rₜ 25.18 min); compound #11, 2″-O-acetyl-deapiopolygalacin D₂ (Rₜ 81.13 min); and compound #12, 2″-O-acetyl-polygalacin D₂ (Rₜ 83.13 min). In the meantime, platyconic acid A proceeded to methylation using diazomethane by the method described in reference #8. As a result, compound #13 (platyconic acid A methyl ester) was obtained.
[Reference 7] Kim, Y. S. et al., Planta Med. 71, 566-568, (2005)
[Reference 8] Choi, Y. H. et al., Molecules 13(11), 2871-2879, (2008)

Structures of the saponin compounds (compounds 1 13) isolated and purified from the roots extract of Platycodon grandiflorum are shown in formula 1 and Table 2.

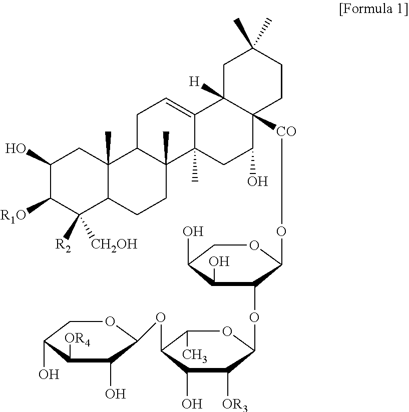

In formula 1, R₁˜R₄ of the saponin compounds (compounds 1-13) isolated and purified from the roots extract of Platycodon grandiflorum are as shown in Table 2.

TABLE 2

| Compound | | | | |
|---|---|---|---|---|
| No. | R₁ | R₂ | R₃ | R₄ |
| 1 | glucopyranosyl-(1→6)- | CH₂OH | H | H |
| | glucopyranosyl-(1→6)- | | | |
| | glucopyranosyl | | | |
| 2 | glucopyranosyl-(1→6)- | CH₂OH | H | apiosyl |
| | glucopyranosyl-(1→6)- | | | |
| | glucopyranosyl | | | |
| 3 | gentiobiosyl | CH₂OH | H | apiosyl |
| 4 | laminaribiosyl | CH₃ | H | apiosyl |
| 5 | glucosyl | CH₃ | H | apiosyl |
| 6 | glucosyl | COOH | H | apiosyl |
| 7 | laminaribiosyl | CH₂OH | H | H |
| 8 | laminaribiosyl | CH₂OH | H | apiosyl |
| 9 | glucosyl | CH₂OH | H | H |
| 10 | glucosyl | CH₂OH | H | apiosyl |
| 11 | laminaribiosyl | CH₃ | acetyl | H |
| 12 | laminaribiosyl | CH₃ | acetyl | apiosyl |
| 13 | glucosyl | COOCH₃ | H | apiosyl |

¹³C-NMR chemical shift (δ) of the saponin compounds (compounds 1-13) isolated and purified from the roots extract of Platycodon grandiflorum are shown in Table 3 and Table 4 (solvent: DMSO-d₆).

TABLE 3

| | 1 | 2 | 3 | 4 | 5 | 6 | 7 | 8 | 9 | 10 | 11 | 12 | 13 |
|---|---|---|---|---|---|---|---|---|---|---|---|---|---|
| carbon No 1 | 45.3 | 45.3 | 45.4 | 44.3 | 44.2 | 46.7 | 45.4 | 45.6 | 45.2 | 45.2 | 44.2 | 44.3 | 45.8 |
| 2 | 68.8 | 68.7 | 67.2 | 70.1 | 70.2 | 69.6 | 67.9 | 70.0 | 69.2 | 70.5 | 70.1 | 70.3 | 69.8 |
| 3 | 88.8 | 88.8 | 89.4 | 83.7 | 83.9 | 83.3 | 89.2 | 85.4 | 86.5 | 86.4 | 83.9 | 84.0 | 84.4 |
| 4 | 48.2 | 48.2 | 48.2 | 42.9 | 42.8 | 56.3 | 48.1 | 48.3 | 48.0 | 48.1 | 42.7 | 42.8 | 56.1 |
| 5 | 47.6 | 47.6 | 45.9 | 48.3 | 48.4 | 49.6 | 45.8 | 48.6 | 48.0 | 48.1 | 48.5 | 48.5 | 50.1 |
| 6 | 19.5 | 19.4 | 20.1 | 18.1 | 18.1 | 20.3 | 19.9 | 19.9 | 19.6 | 19.4 | 18.0 | 18.1 | 20.5 |
| 7 | 33.5 | 33.6 | 34.0 | 33.5 | 33.4 | 33.7 | 34.0 | 34.2 | 33.8 | 33.6 | 33.3 | 33.5 | 33.7 |
| 8 | 40.6 | 40.5 | 41.2 | 40.6 | 40.6 | 40.0 | 41.0 | 41.0 | 40.7 | 40.4 | 40.5 | 40.6 | 40.3 |
| 9 | 45.0 | 45.0 | 48.8 | 48.0 | 48.0 | 47.2 | 48.6 | 47.7 | 48.0 | 47.7 | 47.9 | 48.0 | 47.6 |
| 10 | 38.0 | 38.0 | 38.6 | 37.2 | 37.2 | 37.2 | 38.5 | 38.0 | 37.6 | 37.6 | 37.1 | 37.2 | 37.4 |
| 11 | 24.1 | 24.1 | 24.7 | 24.2 | 24.2 | 24.4 | 24.6 | 24.8 | 24.2 | 24.2 | 24.1 | 24.2 | 24.4 |
| 12 | 123.3 | 123.1 | 123.9 | 123.4 | 123.4 | 122.9 | 123.7 | 123.7 | 123.4 | 123.2 | 123.4 | 123.4 | 123.0 |
| 13 | 144.7 | 144.4 | 145.0 | 144.4 | 144.4 | 144.2 | 144.8 | 144.9 | 144.4 | 144.3 | 144.4 | 144.4 | 144.4 |
| 14 | 42.5 | 42.5 | 43.1 | 42.5 | 42.4 | 42.2 | 42.9 | 43.0 | 42.5 | 42.4 | 42.5 | 42.5 | 42.4 |
| 15 | 36.2 | 36.2 | 36.7 | 36.2 | 36.2 | 36.2 | 36.5 | 36.7 | 36.2 | 36.1 | 36.1 | 36.2 | 36.1 |
| 16 | 73.9 | 74.0 | 73.3 | 74.2 | 74.1 | 73.8 | 73.2 | 74.7 | 74.1 | 73.9 | 74.1 | 74.1 | 74.1 |
| 17 | 49.7 | 49.7 | 50.4 | 50.1 | 50.0 | 49.5 | 50.2 | 50.3 | 50.1 | 49.7 | 50.1 | 50.1 | 50.1 |
| 18 | 41.7 | 41.6 | 42.3 | 41.6 | 41.6 | 41.3 | 42.1 | 42.1 | 41.8 | 41.5 | 41.5 | 41.6 | 41.6 |
| 19 | 47.2 | 47.2 | 47.8 | 47.2 | 47.2 | 47.1 | 47.7 | 47.0 | 47.2 | 47.1 | 47.1 | 47.2 | 47.2 |
| 20 | 31.0 | 31.0 | 31.6 | 30.8 | 30.8 | 30.6 | 31.5 | 31.5 | 30.8 | 30.0 | 30.7 | 30.8 | 30.8 |
| 21 | 36.1 | 36.1 | 36.7 | 36.2 | 36.2 | 35.9 | 36.6 | 36.6 | 36.1 | 36.2 | 36.1 | 36.2 | 36.1 |
| 22 | 32.2 | 32.2 | 32.8 | 31.3 | 31.3 | 31.7 | 32.6 | 32.7 | 31.4 | 32.2 | 31.2 | 31.3 | 31.4 |
| 23 | 63.7 | 63.5 | 64.3 | 66.8 | 66.8 | 63.5 | 63.8 | 64.1 | 63.9 | 63.9 | 66.7 | 66.8 | 64.5 |
| 24 | 67.3 | 67.3 | 67.5 | 14.8 | 14.8 | 181.4 | 67.8 | 65.9 | 66.3 | 66.9 | 14.7 | 14.8 | 175.5 |
| 25 | 19.2 | 19.2 | 19.8 | 17.8 | 17.8 | 16.1 | 19.6 | 18.8 | 18.2 | 18.4 | 17.7 | 17.8 | 15.8 |
| 26 | 17.7 | 17.7 | 18.3 | 17.5 | 17.5 | 17.4 | 18.2 | 18.2 | 17.7 | 17.6 | 17.5 | 17.5 | 17.6 |
| 27 | 27.1 | 27.1 | 27.7 | 27.3 | 27.3 | 27.0 | 27.6 | 27.7 | 27.3 | 27.1 | 27.3 | 27.4 | 27.2 |
| 28 | 176.1 | 176.0 | 176.7 | 175.8 | 175.8 | 175.8 | 176.5 | 176.6 | 175.8 | 176.1 | 175.8 | 175.9 | 175.8 |
| 29 | 33.3 | 33.4 | 34.0 | 33.1 | 33.1 | 33.3 | 33.8 | 33.9 | 33.1 | 33.3 | 33.1 | 33.1 | 33.1 |
| 30 | 24.8 | 24.8 | 25.4 | 25.3 | 25.2 | 24.8 | 25.3 | 25.2 | 25.2 | 24.7 | 25.3 | 25.3 | 25.2 |
| 24-OCH₃ | | | | | | | | | | | | | 51.3 |
| Glu (inner) | | | | | | | | | | | | | |
| 1 | 106.1 | 106.1 | 106.6 | 104.9 | 105.2 | 106.0 | 106.6 | 106.2 | 106.0 | 106.3 | 105.1 | 105.2 | 106.3 |
| 2 | 74.9 | 74.9 | 74.5 | 74.2 | 75.5 | 74.8 | 75.3 | 74.5 | 75.3 | 75.3 | 75.3 | 75.4 | 75.3 |
| 3 | 78.5 | 78.4 | 77.1 | 88.7 | 78.5 | 78.2 | 77.0 | 87.6 | 78.6 | 78.8 | 78.4 | 78.5 | 78.5 |
| 4 | 72.3 | 72.4 | 72.5 | 70.1 | 72.0 | 71.8 | 71.6 | 70.3 | 72.1 | 72.8 | 72.0 | 72.0 | 72.0 |
| 5 | 76.6 | 76.5 | 77.1 | 77.7 | 77.9 | 78.2 | 76.5 | 78.7 | 78.2 | 77.8 | 77.8 | 77.9 | 78.1 |
| 6 | 70.7 | 70.4 | 70.1 | 62.9 | 63.0 | 61.8 | 69.2 | 62.8 | 63.0 | 63.6 | 63.0 | 63.0 | 63.0 |
| Glu (center) | | | | | | | | | | | | | |
| 1 | 105.0 | 105.0 | | | | | | | | | | | |
| 2 | 75.4 | 75.4 | | | | | | | | | | | |
| 3 | 78.4 | 78.5 | | | | | | | | | | | |
| 4 | 71.3 | 71.1 | | | | | | | | | | | |
| 5 | 77.2 | 77.2 | | | | | | | | | | | |
| 6 | 70.2 | 70.2 | | | | | | | | | | | |
| Glu (terminal) | | | | | | | | | | | | | |
| 1 | 105.6 | 105.7 | 105.5 | 105.5 | | | 105.5 | 106.1 | | | | | |
| 2 | 75.2 | 75.2 | 75.5 | 75.3 | | | 74.5 | 76.0 | | | | | |
| 3 | 78.7 | 78.7 | 79.2 | 78.2 | | | 79.2 | 81.1 | | | | | |
| 4 | 70.9 | 71.0 | 71.9 | 72.0 | | | 71.8 | 72.2 | | | | | |
| 5 | 77.7 | 77.7 | 78.5 | 78.2 | | | 78.4 | 79.2 | | | | | |
| 6 | 62.7 | 62.7 | 63.2 | 62.9 | | | 63.1 | 63.1 | | | | | |

TABLE 4

| | 1 | 2 | 3 | 4 | 5 | 6 | 7 | 8 | 9 | 10 | 11 | 12 | 13 |
|---|---|---|---|---|---|---|---|---|---|---|---|---|---|
| Arabinose | | | | | | | | | | | | | |
| 1 | 93.7 | 93.7 | 94.3 | 93.7 | 93.7 | 93.4 | 94.1 | 94.2 | 93.7 | 93.6 | 93.5 | 93.5 | 93.7 |
| 2 | 75.3 | 75.3 | 76.0 | 75.7 | 75.7 | 75.5 | 75.7 | 78.4 | 75.7 | 75.4 | 76.2 | 76.3 | 75.7 |
| 3 | 71.6 | 71.3 | 71.2 | 70.2 | 70.2 | 70.3 | 70.8 | 71.0 | 70.2 | 71.9 | 70.2 | 70.3 | 70.1 |
| 4 | 66.6 | 66.4 | 65.9 | 65.8 | 65.8 | 66.2 | 64.9 | 65.9 | 65.9 | 65.4 | 65.7 | 65.8 | 65.8 |
| 5 | 63.1 | 63.1 | 63.7 | 62.9 | 63.0 | 61.8 | 63.6 | 64.0 | 63.0 | 62.6 | 63.0 | 63.0 | 63.0 |
| Rhamnose | | | | | | | | | | | | | |
| 1 | 101.2 | 101.2 | 101.8 | 101.1 | 101.1 | 101.2 | 101.7 | 101.8 | 101.1 | 101.3 | 98.3 | 98.4 | 101.1 |
| 2 | 71.9 | 72.0 | 72.6 | 72.0 | 72.0 | 71.8 | 72.6 | 72.5 | 72.1 | 71.6 | 73.5 | 73.6 | 72.0 |
| 3 | 72.8 | 72.8 | 72.6 | 72.5 | 72.4 | 72.3 | 72.5 | 73.3 | 72.5 | 72.4 | 70.2 | 70.3 | 72.4 |
| 4 | 84.0 | 83.6 | 84.5 | 83.7 | 83.7 | 83.3 | 84.0 | 85.4 | 83.7 | 83.9 | 83.4 | 83.4 | 83.6 |
| 5 | 68.6 | 68.7 | 69.2 | 68.7 | 68.6 | 68.4 | 69.1 | 69.2 | 68.7 | 68.6 | 68.6 | 68.7 | 68.7 |
| 6 | 18.4 | 18.5 | 19.1 | 18.1 | 18.1 | 18.4 | 18.9 | 19.0 | 18.2 | 18.4 | 18.2 | 18.3 | 18.1 |
| | | | | | | | | | | | 20.7 | 20.7 | |
| | | | | | | | | | | | 170.3 | 170.4 | |
| Xylose | | | | | | | | | | | | | |
| 1 | 106.8 | 106.9 | 107.3 | 106.6 | 106.6 | 106.2 | 107.3 | 107.2 | 106.7 | 106.8 | 106.4 | 106.5 | 106.5 |
| 2 | 76.1 | 76.1 | 75.9 | 75.0 | 75.0 | 74.7 | 76.0 | 75.8 | 75.7 | 75.2 | 75.0 | 75.0 | 75.0 |
| 3 | 84.8 | 78.6 | 85.4 | 85.6 | 85.5 | 85.2 | 79.1 | 84.3 | 78.4 | 84.8 | 78.5 | 85.6 | 85.5 |
| 4 | 69.5 | 71.6 | 69.4 | 39.5 | 69.5 | 68.9 | 71.1 | 70.2 | 71.0 | 69.5 | 69.4 | 69.5 | 69.5 |
| 5 | 67.0 | 67.5 | 67.5 | 66.8 | 66.8 | 66.5 | 66.8 | 67.0 | 67.3 | 66.5 | 67.2 | 66.8 | 66.8 |
| Apiose | | | | | | | | | | | | | |
| 1 | 111.2 | | 111.8 | 111.3 | 111.2 | 110.8 | | 111.7 | | 111.3 | | 111.3 | 111.2 |
| 2 | 77.9 | | 79.1 | 77.9 | 77.9 | 77.8 | | 78.8 | | 77.9 | | 77.9 | 77.9 |
| 3 | 80.5 | | 81.2 | 80.0 | 80.0 | 80.1 | | 84.3 | | 80.5 | | 80.0 | 80.0 |
| 4 | 75.3 | | 75.8 | 75.0 | 75.0 | 74.3 | | 75.7 | | 75.4 | | 75.0 | 75.0 |
| 5 | 65.4 | | 65.9 | 65.8 | 65.8 | 65.1 | | 64.1 | | 64.5 | | 65.8 | 65.8 |

### Example 6

### Hydrolysis of Crude Saponin Obtained from the Roots Extract of Platycodon grandiflorum

### Preparation Example 1

### Acid Hydrolysis of Crude Saponin

20 ml of 5% H₂SO₄ aqueous solution was added to 5 g of the crude saponin obtained in Example 2, followed by reflux for 2 hours. Then, the mixture was cooled down at room temperature and neutralized with 1N NaHCO₃ aqueous solution, followed by extraction using 50 ml of ethyl acetate three times. The extracted ethyl acetate solution was concentrated under reduced pressure, followed by isolation and purification by RP-18 column chromatography (eluant: 60-80% methanol aqueous solution) to give compound #14 (250 mg), #15 (120 mg), #16 (375 mg) and #17 (164 mg). Their chemical structures were identified by spectroscopic data.

### Preparation Example 2

### Alkaline Hydrolysis of Crude Saponin

5 g of the crude saponin obtained in Example 2 was dissolved in 10 ml of 2 N NaOH aqueous solution and 10 ml of 50% methanol aqueous solution, followed by reflux for 5 hours. Then, the mixture was cooled down at room temperature and neutralized with 1N HCl aqueous solution, followed by extraction using 50 ml of ethyl acetate three times. The extracted ethyl acetate solution was concentrated under reduced pressure, followed by isolation and purification by RP-18 column chromatography (eluant: 60-80% methanol aqueous solution) to give compound #18 (120 mg) and #19 (164 mg). Their chemical structures were identified by spectrum data.

Structures of the compounds (compounds 14-19) obtained above are shown in formula 2 and Table 5.

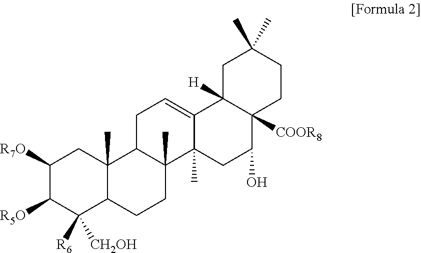

In formula 2, R₅˜R₈ of compounds 14-19 are as shown in Table 5.

TABLE 5

| Compound | | | | |
|---|---|---|---|---|
| No. | R₁ | R₂ | R₃ | R₄ |
| 14 | H | CH₂OH | H | H |
| 15 | H | CH₃ | H | H |
| 16 | H | 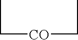 | | H |
| 17 | glucosyl | 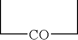 | | H |
| 18 | glucosyl | CH₂OH | H | CH₃ |
| 19 | laminaribiosyl | CH₂OH | H | CH₃ |

¹³C-NMR chemical shift (δ) of the compounds 14-19 are shown in Table 6 (solvent: DMSO-d₆).

TABLE 6

| | sapogenin | | | prosapogenin | | |
|---|---|---|---|---|---|---|
| Carbon No. | 14 | 15 | 16 | 17 | 18 | 19 |
| 1 | 44.6 | 45.8 | 42.0 | 41.4 | 44.0 | 43.9 |
| 2 | 71.7 | 72.5 | 84.6 | 83.5 | 68.5 | 68.6 |
| 3 | 75.1 | 75.6 | 81.8 | 89.5 | 85.4 | 85.4 |
| 4 | 47.1 | 43.3 | 55.2 | 54.5 | 45.8 | 45.8 |
| 5 | 47.9 | 49.1 | 52.1 | 52.2 | 46.5 | 46.5 |
| 6 | 18.9 | 19.2 | 20.1 | 19.7 | 18.1 | 18.1 |
| 7 | 33.7 | 34.2 | 34.0 | 33.8 | 32.5 | 32.5 |
| 8 | 40.0 | 40.9 | 38.2 | 40.6 | 39.1 | 39.0 |
| 9 | 48.7 | 48.5 | 49.1 | 48.5 | 47.0 | 47.0 |
| 10 | 37.1 | 38.1 | 36.9 | 38.0 | 36.4 | 36.3 |
| 11 | 24.1 | 24.9 | 25.2 | 25.0 | 23.1 | 23.1 |
| 12 | 122.4 | 123.4 | 121.3 | 122.2 | 121.9 | 122.0 |
| 13 | 145.0 | 146.0 | 147.3 | 146.2 | 143.3 | 143.4 |
| 14 | 42.1 | 43.1 | 43.1 | 42.7 | 41.1 | 41.1 |
| 15 | 35.9 | 36.9 | 36.7 | 36.9 | 34.9 | 34.9 |
| 16 | 74.6 | 73.9 | 75.9 | 75.0 | 73.3 | 73.3 |
| 17 | 49.6 | 49.7 | 50.8 | 49.3 | 48.0 | 48.0 |
| 18 | 41.3 | 42.3 | 40.9 | 41.9 | 40.4 | 40.4 |
| 19 | 46.5 | 48.1 | 48.1 | 47.7 | 45.4 | 45.6 |
| 20 | 30.9 | 31.9 | 28.3 | 30.3 | 29.8 | 29.8 |
| 21 | 36.1 | 37.0 | 34.2 | 36.8 | 34.9 | 34.9 |
| 22 | 32.8 | 33.7 | 31.5 | 31.5 | 31.5 | 31.5 |
| 23 | 63.9 | 68.5 | 58.3 | 57.5 | 62.5 | 62.5 |
| 24 | 64.5 | 15.4 | 179.5 | 178.6 | 64.1 | 64.1 |
| 25 | 17.5 | 18.4 | 18.3 | 17.7 | 17.2 | 17.1 |
| 26 | 17.5 | 18.2 | 19.1 | 18.4 | 16.2 | 16.2 |
| 27 | 27.1 | 28.1 | 26.9 | 27.7 | 26.1 | 26.1 |
| 28 | 180.0 | 180.9 | 185.0 | 180.4 | 176.8 | 176.8 |
| 29 | 33.2 | 34.2 | 31.7 | 33.3 | 32.2 | 32.2 |
| 30 | 24.6 | 25.6 | 25.7 | 25.2 | 23.5 | 23.5 |
| 24-OCH₃ | | | | | 50.8 | 50.8 |
| Glucose(inner) | | | | | | |
| 1 | | | | 105.7 | 105.3 | 104.6 |
| 2 | | | | 75.7 | 74.2 | 73.0 |
| 3 | | | | 79.2 | 77.7 | 87.5 |
| 4 | | | | 71.8 | 70.5 | 68.6 |
| 5 | | | | 78.8 | 77.6 | 77.1 |
| 6 | | | | 63.0 | 61.5 | 61.1 |
| Glucose(terminal) | | | | | | |
| 1 | | | | | | 104.7 |
| 2 | | | | | | 74.5 |
| 3 | | | | | | 77.7 |
| 4 | | | | | | 70.5 |
| 5 | | | | | | 77.3 |
| 6 | | | | | | 61.4 |

### Experimental Example 1

### HCV RNA Replication Inhibitory Activity in HCV Replicon (Subgenomic RNA Replicon) Cell Line

HCV RNA replication inhibitory activity in HCV replicon cell line of saponin components of Platycodon grandiflorum, the roots extract of Platycodon grandiflorum and the composition containing the same of the present invention and sapogenin and prosapogenin was investigated by the following experiments.

Culture of HCV RNA Replicon Cell Line

To screen a compound that is capable of inhibiting HCV replication, each compound was added to Huh-7 human hepatoma cell line harboring HCV RNA replicon, followed by culture. Then, expression level of HCV RNA was quantified and its inhibitory activity was measured. HCV replicon used in this invention was derived from HCV-1b Hepatitis C virus gene that was bicistronic replicon composed of HCV IRES, neomycin resistant gene, EMCV (encephalomyocarditis virus) IRES. HCV non-structural proteins were composed of the sequences comprising NS3-NS5B and HCV 3′end. An expression vector harboring HCV replicon proceeded to in vitro transcription, and the obtained HCV replicon was transfected into the Huh-7 cells by electroporation. To select only those cells having HCV replicon, Huh-7 cells were cultured with medium containing the antibiotic G418 (500 μg/ml). The selected cells were cultured with DMEM (Dulbecco's modified Eagles media) containing 10% FBS, non-essential amino acids and 500 μg/ml of G418.

HCV RNA Replication Inhibitory Activity in HCV Replicon Cells of Compounds

Huh-7 cells harboring HCV subgenomic RNA replicon were cultured overnight in 6 well plate (3×10⁵ cells/well), at 37° C. and 5% CO₂ with DMEM containing 10% FBS, non-essential amino acids and 500 μg/ml of G418. Medium of each well was replaced with DMEM containing 2% FBS, non-essential amino acids and 500 μg/ml of G418. Test compound was dissolved in DMSO, which was added to each well at different concentrations, followed by culture in a 5% CO₂ incubator at 37° C. for 72 hours. Equal amount of DMSO (negative control) and Interferon-α (positive control) were added as controls. Upon completion of the culture, medium of each well was eliminated, followed by washing with 1 ml of PBS. 250 μl/well of trypsin/EDTA was added thereto and cells were separated from the plate and washed with PBS again to eliminate medium. Total RNA was isolated from the cells by using SV total RNA isolation system (Promega corporation), followed by quantification using GeneQuant pro (Amersham bioscience). EC₅₀ against HCV replicon of each compound was investigated by RT-PCR and the result was compared with those of controls. RT-PCR was performed with the primer targeting HCV 1b NS5B region and by AccessQuick™ RT-PCR system (Promega corporation). To obtain more accurate EC₅₀ values, quantitative real-time PCR was performed along with RT-PCR. cDNA was obtained from the isolated RNA by using Reverse transcription system (Promega corporation), followed by quantitative real-time PCR using iQ SYBR Green Supermix (Bio-rad). At the same time, one-step real time RT-PCR was performed using Taqman probe to investigate the inhibitory activity of each compound. At this time, the primer targeting HCV 5′-UTR was used and GAPDH (Glyceraldehyde-3-phosphate dehydrogenase) gene was used as a reference gene for correction. Real time RT-PCR was performed by using iCycler iQ5 system (Bio-rad). The EC₅₀ value was calculated by iCycler iQ5 optical system software (Bio-rad) program to determine the inhibitory activity. HCV replicon inhibitory activity of saponin components of Platycodon grandiflorum, the roots extract of Platycodon grandiflorum and the composition containing the same of the present invention and sapogenin and prosapogenin is shown in Table 7 and Table 8.

TABLE 7
HCV RNA replication inhibitory activity of the
roots extract of Platycodon grandiflorum

| Sample | Water Extract | | Ethanol Extract | | Methanol Extract | |
|---|---|---|---|---|---|---|
| (Molecular | Sample | EC₅₀ | Sample | EC₅₀ | Sample | EC₅₀ |
| Weight) | No. | μg/ml | No. | μg/ml | No. | μg/ml |
| Total Extract | DrJ-1 | 3-7 | DrJ-2 | 3-8 | DrJ-3 | 3-7 |
| Crude Saponin | DrJ-9 | 0.1-0.2 | DrJ-10 | 0.3 | DrJ-11 | 0.1-0.3 |
| less than 100,000 | DrJ-12 | 2-3 | DrJ-16 | 2 | DrJ-20 | 4 |
| less than 5,000 | DrJ-13 | 2-5 | DrJ-17 | 2-3 | DxJ-21 | 2-3 |
| 5,000~1,000 | DrJ-14 | 0.1-0.3 | DrJ-18 | 0.2 | DrJ-22 | 0.2-0.3 |
| 100,000~1,000 | DrJ-15 | 0.7-1 | DrJ-19 | 0.7 | DrJ-23 | 0.8 |
| 5,000~500 | DrJ-24 | 0.1-0.2 | DrJ-25 | 0.1-0.2 | DrJ-26 | 0.1-0.2 |

TABLE 8
HCV RNA replication inhibitory activity of saponin components of
Platycodon grandiflorum

| Compound | | Inhibitory Activity |
|---|---|---|
| No. | Compound Name | EC₅₀ (μg/ml) |
| 1 | deapioplatycoside E | >10 |
| 2 | platycoside E | 0.7 |
| 3 | Platycodin D₃ | 0.2 |
| 4 | polygalacin D₂ | >10 |
| 5 | polygalacin D | 0.8 |
| 6 | platyconic acid A | 0.2 |
| 7 | deapioplatycodin D₂ | 0.2 |
| 8 | platycodin D₂ | 0.1 |
| 9 | deapioplatycodin D | 0.2 |
| 10 | platycodin D | 0.1 |
| 11 | 2″-O-acetyl-deapiopolygalacin D₂ | >10 |
| 12 | 2″-O-acetyl-polygalacin D₂ | 1 |
| 13 | platyconic acid A methyl ester | 5 |
| 14 | platycodigenin | >10 |
| 15 | polygalacic acid | 2 |
| 16 | platycogenic acid A lactone | 0.4 |
| 17 | platycogenic acid A lactone | 2 |
| | 3-O-glucopyranoside | |
| 18 | platycodigenin 3-O-glucopyranoside | >10 |
| | 28-methyl ester | |
| 19 | platycodigenin 3-O-laminaribioside | >10 |
| | 28-methyl ester | |

### Experimental Example 2

### HCV RNA Replication Inhibitory Effect by Combination Therapies of Interferon with the Roots Extract of Platycodon grandiflorum and Saponin Components of Platycodon grandiflorum

Following experiments were performed to investigate drug interaction related to HCV replication inhibition of combination therapies of saponin components of Platycodon grandiflorum, the roots extract of Platycodon grandiflorum and compositions comprising Platycodon grandiflorum saponin with Interferon-α.

The same HCV sub-genomic replicon cells as the one used in Experimental Example 1 was used. Human Interferon α-A (PBL Biomedical Laboratories) was used. To calculate EC₅₀ value of roots extract of Platycodon grandiflorum and Interferon-α, they were added to HCV replicon cells at different concentrations, followed by measurement of EC₅₀ by the same manner as described in Experimental Example 1. To investigate drug interaction of combination therapy, roots extract of Platycodon grandiflorum and Interferon-α were treated to HCV replicon cells independently or together at a required concentration, followed by culture in a 5% CO₂ incubator at 37° C. for 3 days-3 weeks. Then, HCV replication inhibitory effect was measured. To determine drug interaction of combination therapy, the inhibitory activity observed from combination therapy was compared with that observed from single treatment at a required concentration and calculated by the method of Chou (Chou, T. C., 2006, Theoretical basis, experimental design, and simulation of synergism and antagonism in drug combination studies. Pharmacological Reviews. 58:621-681). Drug interaction was presented as synergy, additive effect and antagonism. And the results of drug interaction of saponin components of Platycodon grandiflorum, the roots extract of Platycodon grandiflorum and compositions comprising Platycodon grandiflorum saponin and interferon on HCV replication inhibition are as shown in Tables 9, 10 and 11. CI (combination index) values of drug interaction were calculated using CalcuSyn program (Biosoft). CI lower than 1 represents synergic effect, CI of about 1 represents additive effect, and CI higher than 1 represents antagonism. Platycodon grandiflorum extract DrJ-14 and DrJ-24, and Platycodon grandiflorum crude saponin DrJ-9 were treated at concentration of 0.94 μg/ml, 1.88 μg/ml, 3.75 μg/ml, 7.50 μg/ml, 15 μg/ml and 30 μg/ml, and Interferon was combined treated at concentration of 0.47 U/ml, 0.94 U/ml, 1.88 U/ml, 3.75 U/ml, 7.5 U/ml and 15 U/ml. In this combined treatment experiment of Platycodon grandiflorum extract DrJ-14 and DrJ-24, and Platycodon grandiflorum crude saponin DrJ-9 with interferon, combined treatment was evaluated to have synergic effect since all the combination indices show values lower than 1.

TABLE 9
Synergic effect on HCV RNA replication inhibitory effect of
Platycodon grandiflorum extract DrJ-14 and interferon

| | |
|---|---|
| | CI (Combination Index) |
| DrJ-14 (μg/ml)/IFN (U/ml) | at HCV inhibition of |

| ratio | EC₅₀ | EC₇₅ | EC₉₀ |
|---|---|---|---|
| 2:1 | 0.77 | 0.36 | 0.24 |
| 4:1 | 0.90 | 0.40 | 0.24 |
| 8:1 | 0.90 | 0.52 | 0.37 |
| 16:1 | 0.99 | 0.65 | 0.48 |

TABLE 10
Synergic effect on HCV RNA replication inhibitory effect of
Platycodon grandiflorum extract DrJ-24 and interferon

| | |
|---|---|
| | CI (Combination Index) |
| DrJ-24 (μg/ml)/IFN (U/ml) | at HCV inhibition of |

| ratio | EC₅₀ | EC₇₅ | EC₉₀ |
|---|---|---|---|
| 2:1 | 0.70 | 0.31 | 0.22 |
| 4:1 | 0.80 | 0.34 | 0.21 |
| 8:1 | 0.80 | 0.43 | 0.30 |
| 16:1 | 0.88 | 0.53 | 0.38 |

TABLE 11
Synergic effect on HCV RNA replication inhibitory effect of
Platycodon grandiflorum crude saponin DrJ-9 and interferon

| | |
|---|---|
| | CI (Combination Index) |
| DrJ-9 (μg/ml)/IFN (U/ml) | at HCV inhibition of |

| ratio | EC₅₀ | EC₇₅ | EC₉₀ |
|---|---|---|---|
| 2:1 | 0.61 | 0.42 | 0.32 |
| 4:1 | 0.73 | 0.48 | 0.34 |
| 8:1 | 0.96 | 0.60 | 0.39 |
| 16:1 | 1.26 | 0.70 | 0.40 |

### Experimental Example 3

### Cytotoxicity Test

To confirm cytotoxicity of saponin components of Platycodon grandiflorum, the roots extract of Platycodon grandiflorum and the composition containing the same of the present invention and saponin analogues, in vitro MTT assay was performed with HepG2 cells. As a result, the saponin components of Platycodon grandiflorum, the roots extract of Platycodon grandiflorum and the composition containing the same of the present invention and saponin analogues were evaluated to be safe substances since their estimated CC₅₀ values were much greater than 100 μg/ml.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A pharmaceutical composition for preventing or treating Hepatitis C comprising one or more active ingredients selected from the group consisting of the roots extract of Platycodon grandiflorum extracted using water, an organic solvent or a mixture thereof; and saponin components isolated from the roots extract of Platycodon grandiflorum.

2. The pharmaceutical composition for preventing or treating Hepatitis C according to claim 1, wherein the organic solvent is 10-100% concentration of C₁-C₄ lower alcohol.

3. The pharmaceutical composition for preventing or treating Hepatitis C according to claim 1, wherein the Platycodon grandiflorum extract includes the Platycodon grandiflorum extract prepared from the solvent-extracted Platycodon grandiflorum extract by filtering with ultra-filtration membrane.

4. The pharmaceutical composition for preventing or treating Hepatitis C according to claim 3, the ultra-filtration membrane has the molecular weight cut-off of 100,000, 5,000 or 1,000.

5. The pharmaceutical composition for preventing or treating Hepatitis C according to claim 3, wherein the Platycodon grandiflorum extract prepared by filtering with ultra-filtration membrane has the molecular weight of 1,000-100,000 or 1,000-5,000.

6. The pharmaceutical composition for preventing or treating Hepatitis C according to claim 3, wherein the Platycodon grandiflorum extract includes the Platycodon grandiflorum extract having the molecular weight of 500-5,000 prepared by nano-filtration membrane with molecular weight cut-off up to 500 from the Platycodon grandiflorum extract prepared by ultra-filtration membrane.

7. The pharmaceutical composition for preventing or treating hepatitis C according to claim 1, wherein the saponin components isolated from the roots extract of Platycodon grandiflorum include the saponin compound of formula 1 and the (pro)sapogenin compound of formula 2: 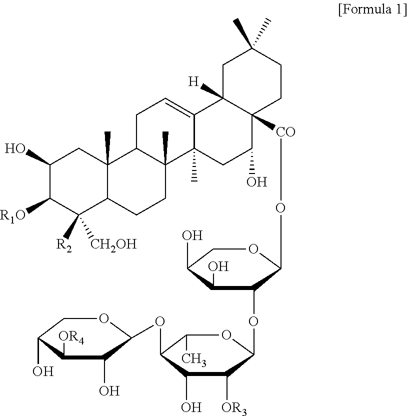
In formula 1,
R₁ is glucopyranosyl-(1→6)-glucopyranosyl-(1→6)-glucopyranosyl 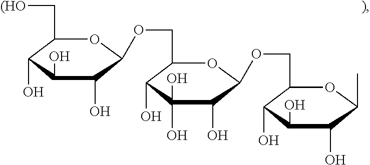
gentiobiosyl 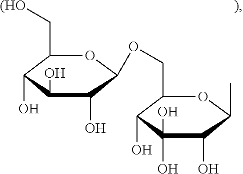
glucosyl 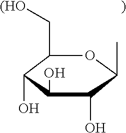
or laminaribiosyl 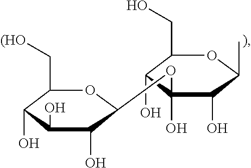
R₂ is CH₂OH, CH₃, COOH or COOCH₃,
R₃ is H or acetyl,
R₄ is H or apiosyl 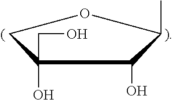 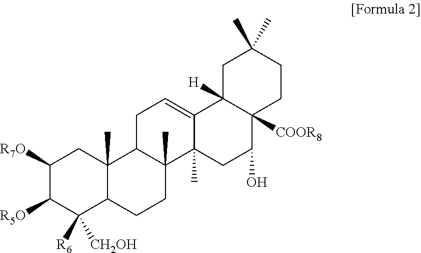
In formula 2,
R₅ is H, glucosyl or laminaribiosyl,
R₆ is CH₂OH or CH₃,
R₇ is H,
R₆ and R₇ can be —CO— linked each other,
R₈ is H or CH₃.

8. The pharmaceutical composition for preventing or treating Hepatitis C according to claim 1, wherein the saponin components isolated from the roots extract of Platycodon grandiflorum include Platycodon grandiflorum crude saponin obtained by the processes of isolating Platycodon grandiflorum solvent extract by using reverse phase gel; dissolving the obtained extract in ethanol or methanol; and crystallizing the resultant with ethyl acetate.

9. The pharmaceutical composition for preventing or treating Hepatitis C according to claim 7, wherein the saponin compound of formula 1 is selected from the group consisting of deapioplatycoside E, platycoside E, platycodin D₃, polygalacin D₂, polygalacin D, platyconic acid A, deapioplatycodin D₂, platycodin D₂, deapioplatycodin D, platycodin D, 2″-O-acetyl-deapiopolygalacin D₂, 2″-O-acetyl-polygalacin D₂ and platyconic acid A methyl ester.

10. The pharmaceutical composition for preventing or treating Hepatitis C according to claim 7, wherein the (pro)sapogenin compound of formula 2 is selected from the group consisting of platycodigenin, polygalacic acid, platycogenic acid A lactone, platycogenic acid A lactone 3-O-glucopyranoside, platycodigenin 3-O-glucopyranoside 28-methyl ester and platycodigenin 3-O-laminaribioside 28-methyl ester.

11. The pharmaceutical composition for preventing or treating Hepatitis C according to claim 10, wherein the (pro)sapogenin compound is prepared by hydrolysis of crude saponin of Platycodon grandiflorum.

12. The pharmaceutical composition for preventing or treating Hepatitis C according to claim 1, wherein the composition further comprises one or more HCV proliferation inhibitors.

13. The pharmaceutical composition for preventing or treating Hepatitis C according to claim 12, wherein the HCV proliferation inhibitor is selected from the group consisting of immune modulator, cell signaling regulator, antiviral agent, HCV polymerase (NS5B) inhibitor, HCV protease (NS3/4A) inhibitor, HCV helicase (NS3 helicase) inhibitor, HCV NS4B inhibitor, HCV NS5A inhibitor, HCV cell entry inhibitor and HCV assembly inhibitor.

14. The pharmaceutical composition for preventing or treating Hepatitis C according to claim 1, wherein the composition further comprises interferon which is the immune modulator; and Ribavirin.

15. The pharmaceutical composition for preventing or treating Hepatitis C according to claim 13, wherein the immune modulator is selected from the group consisting of natural interferon, Interferon-α, Interferon-β, Interferon-γ, pegylated-Interferon, albumin-linked Interferon and cytokine.

16. A health functional food for the prevention or improvement of Hepatitis C comprising one or more active ingredients selected from the group consisting of the roots extract of Platycodon grandiflorum extracted using water, an organic solvent or a mixture thereof; and saponin components isolated from the roots extract of Platycodon grandiflorum.
